(19) United States
(12) Patent Application Publication
(10) Pub. No.: US 2016/0045190 A1
(43) Pub. Date: Feb. 18, 2016

(54) ENDOSCOPIC BIOPSY ONE-WAY TRAP

(71) Applicant: Boston Scientific Scimed, Inc., Maple Grove, MN (US)

(72) Inventors: Justin ELFMAN, Cherry Hill, NJ (US); Daniel FAULKNER, Cambridge, MA (US); Paul SMITH, Smithfield, RI (US)

(21) Appl. No.: 14/811,427

(22) Filed: Jul. 28, 2015

Related U.S. Application Data
(60) Provisional application No. 62/036,404, filed on Aug. 12, 2014.

(51) Int. Cl. A61B 10/02; A61B 10/04

(52) U.S. Cl. CPC A61B 10/0266; A61B 10/04; A61B 2010/045

(57) ABSTRACT

A device for obtaining a tissue sample includes a needle body extending along a longitudinal axis from a proximal end to a distal end and including a lumen extending therethrough and a plurality of flaps coupled to the needle body via hinges. Each of the flaps is movable between a closed configuration in which a cutting end of each of the flaps extends towards the longitudinal axis of the needle body so that the flaps extend radially inward to at least partially cover a distal opening of the lumen, and an open configuration, in which the flaps are rotated about the hinges so that cutting ends thereof are moved toward an interior surface of the lumen to uncover the distal opening of the lumen and permit a target tissue to be received therebetween and into the lumen.

### PRIORITY CLAIM

The present application claims priority to U.S. Provisional Patent Application Ser. No. 62/036,404 filed, Aug. 12, 2014; the disclosure of which is incorporated herewith by reference.

### BACKGROUND

Needle biopsy procedures are common for the diagnosis and the staging of disease. For example, a fine needle aspiration needle may be advanced through a working channel of an endoscope to a target tissue site. Although fine needle aspiration is a highly sensitive and specific procedure, it is often difficult to acquire a suitable sample under certain clinical situations. The more cells or tissue that can be acquired, the greater the potential for a definitive diagnosis. Larger gauge needles, however, may be difficult to pass along tortuous paths through anatomy to target sites and may acquire samples including more blood, making it more difficult to obtain a diagnosis.

### SUMMARY

The present disclosure is directed to a device for obtaining a tissue sample, comprising a needle body extending along a longitudinal axis from a proximal end to a distal end and including a lumen extending therethrough and a plurality of flaps coupled to the needle body via hinges, each of the flaps movable between a closed configuration in which a cutting end the flaps extends towards the longitudinal axis of the needle body so that the flaps extend radially inward to at least partially cover a distal opening of the lumen, and an open configuration, in which the flaps are rotated about the hinges so that cutting ends thereof are moved toward an interior surface of the lumen to uncover the distal opening of the lumen and permit a target tissue to be received therebetween and into the lumen.

The plurality of flaps may be biased toward the closed configuration.

The hinges may be living hinges.

The plurality of flaps may be coupled to the distal end of the needle body.

The interior surface of the lumen may include a recess extending therein, the recess sized and shaped to receive one of the plurality of flaps, when the plurality of flaps is in the open configuration.

The recess may be a circumferential groove sized to receive all of the plurality of flaps, when the plurality of flaps is in the open configuration.

The cutting end of each of the plurality of flaps may be sharpened to cut the target tissue from a surrounding tissue, trapping the target tissue in the lumen.

The plurality of flaps may be formed via a tab cut through a wall defining the needle body and bent toward the longitudinal axis of the needle body.

The plurality of flaps may be pivotally coupled to a distal portion of the needle body.

The device may further comprise an oversheath longitudinally slidable over the needle body to move the plurality of flaps between the closed and open configurations.

The present disclosure is also directed to a system for collecting a tissue sample comprising a device as described above and a tissue removal tool coupleable to the distal end of the needle to remove the target tissue from within the lumen of the needle, the tissue removal tool including arms at a proximal end thereof for holding the plurality of flaps in the open configuration when the tissue removal tool is coupled to the needle.

The present disclosure is also directed to a system for collecting a tissue sample, comprising a needle extending longitudinally from a proximal end to a distal end, the needle including a lumen extending therethrough and a plurality of flaps coupled thereto and movable between a closed configuration and an open configuration, each of the plurality of flaps extending from a first end coupled to the distal end via a hinge biasing the plurality of flaps in the closed configuration to a second end, the second end extending toward a longitudinal axis of the needle so that the plurality of flaps extend radially inward to cover a distal opening of the lumen in the closed configuration, the plurality of flaps adapted for rotating about the hinges via which the plurality of flaps is connected to the needle body so that second ends thereof are moved toward an interior surface of the lumen to uncover the distal opening of the lumen and permit a target tissue to be received therebetween in the open configuration and a tissue removal tool coupleable to the distal end of the needle to remove the target tissue from within the lumen of the needle, the tissue removal tool including arms at a proximal end thereof for holding the plurality of flaps in the open configuration when the tissue removal tool is coupled to the needle.

The tissue removal tool may include a channel extending therethrough, the channel substantially aligning with the lumen of the needle when the tissue removal tool is coupled thereto.

The plurality of flaps may be coupled to the distal end of the needle via a living hinge.

The interior surface of the lumen may include a recess extending therein, the recess sized and shaped to receive at least one of the plurality of flaps, when the plurality of flaps is in the open configuration.

The cutting ends of the plurality of flaps may be sharpened to cut the target tissue from a surrounding tissue, trapping the target tissue in the lumen.

The present disclosure is also directed to a method for collecting a tissue sample, comprising inserting a needle to a target tissue within a patient body via a working channel of an endoscope, the needle extending longitudinally from a proximal end to a distal end and including a lumen extending therethrough, inserting the distal end of the needle into the target tissue such that a portion of the target tissue moves a plurality of flaps coupled to a distal portion of the needle from a closed configuration, in which cutting ends of the plurality of flaps extend toward a longitudinal axis of the needle to cover a distal opening of the lumen, to an open configuration, in which the plurality of flaps rotate about hinges connecting the plurality of flaps to the needle to uncover the distal opening and permit the target tissue to be received therein, and moving the needle proximally relative to the target tissue such the plurality of flaps is moved toward the closed configuration and the cutting ends of the plurality of flaps cut the portion of the target tissue received within the lumen from a surrounding portion of the target tissue, trapping a tissue sample therein.

### BRIEF DESCRIPTION

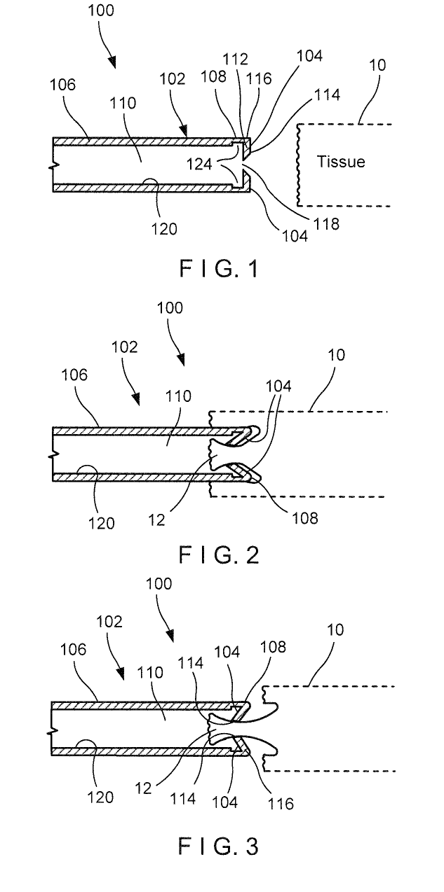

FIG. 1 shows a longitudinal cross-sectional view of a distal portion of a device according to an exemplary embodiment of the present disclosure;

FIG. 2 shows another longitudinal cross-sectional view of the device of FIG. 1;

FIG. 3 shows another longitudinal cross-sectional view of the device of FIG. 1;

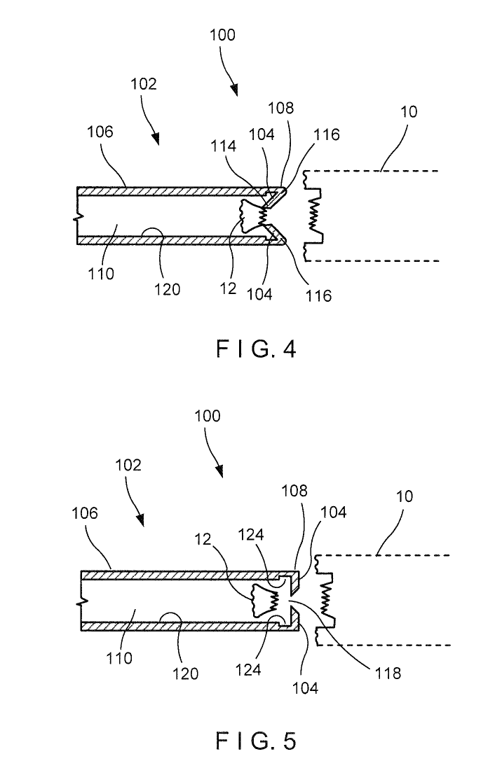

FIG. 4 shows yet another longitudinal cross-sectional view of the device of FIG. 1;

FIG. 5 shows another longitudinal cross-sectional view of the device of FIG. 1;

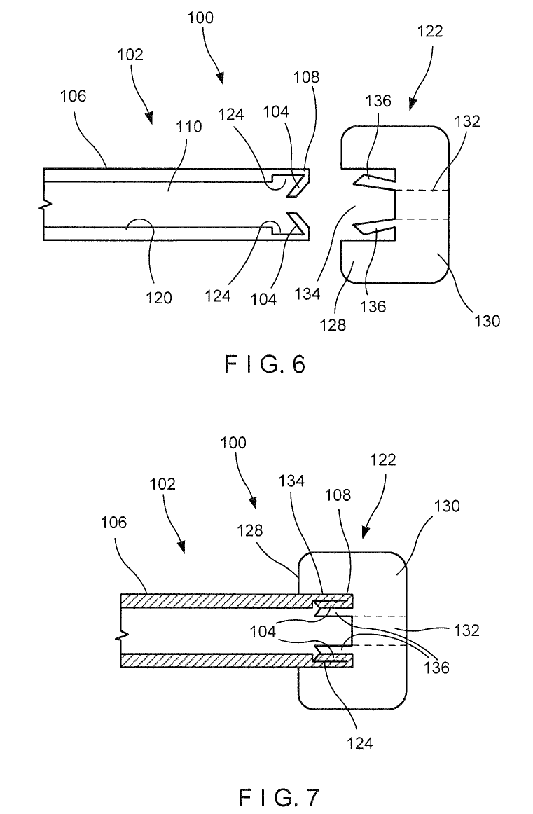

FIG. 6 shows a side view of a tissue release tool for the device of FIG. 1;

FIG. 7 shows a side view of the tissue release tool of FIG. 6, engaging the device of FIG. 1;

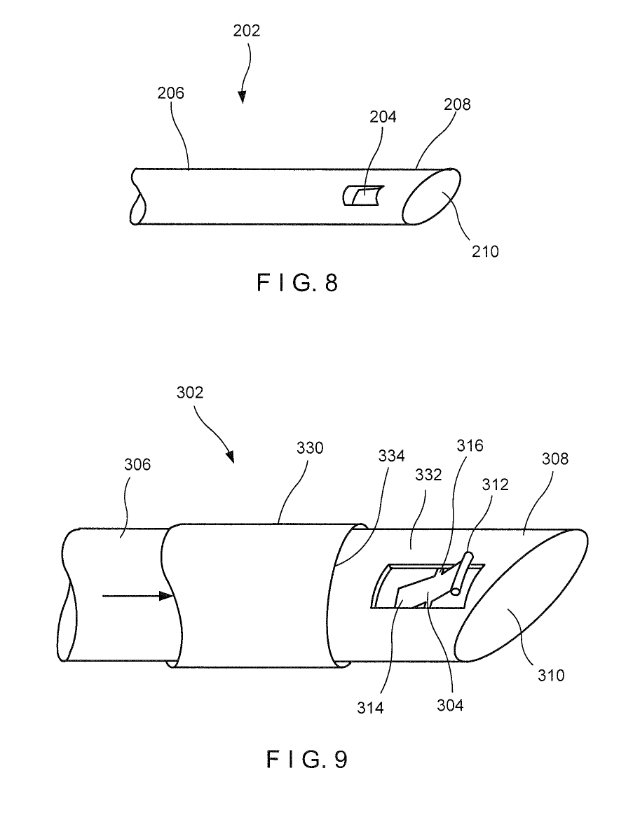

FIG. 8 shows a perspective view of a device according to another exemplary embodiment of the present disclosure; and

FIG. 9 shows a perspective view of a device according to yet another exemplary embodiment of the present disclosure.

### DETAILED DESCRIPTION

The present disclosure may be further understood with reference to the following description and the appended drawings, wherein like elements are referred to with the same reference numerals. The present disclosure relates to endoscopic devices and, in particular, devices for obtaining tissue samples. Exemplary embodiments of the present disclosure describe a needle comprising flaps on opposing sides of the needle and extending radially inward. The flaps are movable between a closed configuration covering a distal opening of a lumen extending through the needle and an open configuration opening the distal opening of the lumen to receive a tissue sample within the lumen. The flaps are configured to cut the tissue sample from surrounding tissue and trap the tissue sample within the lumen to be removed from the patient's body. It should be noted that the terms “proximal” and “distal” as used herein, are intended to refer to a direction toward (proximal) and away from (distal) a user of the device.

As shown in FIGS. 1-7, a system 100 for collecting a tissue sample according to an exemplary embodiment of the present disclosure comprises a needle 102 including opposed flaps 104 at a distal end 108 of a needle body 106 for cutting a tissue sample 12 from a surrounding tissue area 10 and trapping the tissue sample 12 within a lumen 110 of the needle 102. The flaps 104 extend from a first end 112 connected to the distal end 108 via a hinge 116 to a second end 114 which extends inward toward a longitudinal axis of the needle 102. The flaps 104 are movable between a closed configuration, in which the flaps 104 extend radially inward to cover a distal opening 118 of the lumen 110, and an open configuration, in which the flaps 104 move proximally about the hinge 108 so that the second ends 114 extend toward an interior surface 120 of the lumen 110, uncovering the distal opening 118 of the lumen 110 to permit a tissue sample to be received therethrough. In this embodiment, two flaps 104 are positioned at the distal opening 118 however, as would be understood by those skilled in the art, a single flap 118 or any number of flaps 118 may be used as desired. In particular, as the distal end 108 of the needle 102 is inserted into the target tissue 10, the flaps 104 move from the closed configuration to the open configuration to permit the tissue sample 12 to be received therebetween and into the lumen 110. Once the tissue sample 12 has been received therein, a user may move the needle 102 proximally relative to the tissue area 10, causing the second ends 114 of the flaps 104 to cut the tissue sample 12 from the surrounding tissue area 10. The flaps 104 in this embodiment are biased toward the closed configuration so that, when the tissue sample 12 is separated from the tissue area 10, the flaps 104 revert to the closed configuration to trap the tissue sample 12 within the lumen 110. As shown specifically in FIGS. 6 and 7, the system 100 may further comprise a tissue removal tool 122 for removing the tissue sample 12 from the needle 102, once the needle 102 has been removed from the patient body.

The needle body 106 extends longitudinally from a proximal end (not shown) to the distal end 108 and includes the lumen 110 extending therethrough along the longitudinal axis thereof. The needle body 106 may be sized and shaped to be received through a working channel of an endoscope for insertion therethrough into a living body, for example, under ultrasound guidance. The needle body 106 should be sufficiently flexible to be passed through the same tortuous paths of the body accessible by an endoscope or other flexible insertion instrument. The distal end 108 may be tapered or beveled to facilitate insertion to the target tissue area 10. Although the exemplary embodiments show and describe the flaps 104 as connected to the distal end 108, it will be understood by those of skill in the art that the flaps 104 may be positioned within the lumen 110, slightly proximally of the distal end 108 to facilitate insertion of the distal end 108 into the target tissue area 10. Where the flaps 104 are positioned at the distal end 108, however, the interior surface 120 may include a recess 124 along a portion thereof, immediately proximal of the flaps 104, for receiving the flaps 104 when the flaps 104 are moved to the closed configuration. In one embodiment, the recess 124 may be configured as a circumferential groove. In another embodiment, the needle 102 may include more than one recess 124, each of which corresponds to one of the flaps 104. The recess 124 receiving the flaps 104 creates a sharper cutting edge at the distal end 108, facilitating insertion thereof into the target tissue 10.

Each of the flaps 104 extends from a first end 112 connected to the needle body 106 to a second end 114. The flaps 104 in this embodiment are biased toward a position in which the second ends 114 extend toward a longitudinal axis of the needle 102 to close the lumen 110. In one exemplary embodiment, the needle 102 includes two flaps 104, each positioned on opposing sides of the needle body 106. It will be understood by those of skill in the art, however, that although the needle 102 is described and shown including a pair of flaps 104, the needle 102 may include any number of flaps 104. For example, the needle 102 may include a single flap 104 or more than two flaps. The flaps 104 are sized and shaped to fit the contours of the needle body 106. In particular, the flaps 104 may be sized and shaped so that, when they are in the closed configuration, the flaps 104 cover the distal opening 126 of the lumen 110. Each of the flaps 104 is connected to the distal end 108 via a hinge 116, which may be configured as, for example, a living hinge. The living hinges may be cut and formed using standard laser-cutting and stamping techniques known in the art. Although exemplary embodiments show and describe the hinges 116 as living hinges, it will be understood by those of skill in the art that any or all of the hinges 116 may be formed as any of a variety of types of hinge joints so long as the flaps 104 are able to pivot thereabout between the closed and open configurations. As described above, the flaps 104 may be positioned at the distal end 108 of the needle body 106 or immediately proximally thereto. The second ends 114 may be sharpened to facilitate a cutting of the tissue sample 12 from the surrounding target tissue 10. In the closed configuration, the flaps 104 may extend substantially perpendicular to the longitudinal axis of the needle body 106. In another embodiment, the flaps 104 may extend at an angle relative to the longitudinal axis, the second ends 114 pointed toward the proximal end of the needle body 106, in the closed configuration.

As shown in FIGS. 6-7, the tissue removal tool 122 is coupleable to the distal end 108 of the needle 102 to move the flaps 104 from the closed configuration (toward which they are biased) to the open configuration to aid in removal of the tissue sample 12 from the lumen 110 of the needle 102. The tissue removal tool 122 extends from a proximal end 128 to a distal end 130 and includes a channel 132 extending therethrough so that when the proximal end 128 is coupled to the distal end 108 of the needle 102, the channel 132 is substantially aligned with the lumen 110 of the needle 102. The proximal end 128 includes a recess 134 substantially aligned with the channel 132. The recess 134 is sized and shaped to receive the distal end 108 of the needle 102 and includes arms 136 extending proximally from a distal end 138. The arms 136 are configured to move the flaps 104 to the open configuration and to hold the arms 136 in the open configuration while the tissue removal tool 122 is mounted to the distal end 108 of the needle 102. Thus the tissue sample 102 may be removed from the lumen 110 via the channel 132.

According to an alternate embodiment of the tissue removal tool 122, rather than arms 136 for holding the flaps 104 in the open configuration, the tissue removal tool 122 may include features for bending and/or breaking one or more of the hinges 116 connecting the flaps 104 to the needle body 106 to provide access to the lumen 110 for sample removal.

Other embodiments of the needle 102, however, may not require use of the tissue removal tool 122 to remove the tissue sample. In one exemplary embodiment, a distal portion of the needle 102 including the flaps 104 is detachable from a remaining portion of the needle. In one example, the distal portion of the needle 102 is coupled to a proximal portion of the needle 102 via a frangible link designed to fail when subject to a predetermined load. Thus, the distal portion may be snapped off by breaking the frangible link once the needle 102 has been removed from the body with a tissue sample captured therein. In another example, the distal portion of the needle 102 is configured as a detachable cap coupled to a proximal portion of the needle 102 via a manual release mechanism. Thus, the distal portion of the needle 102 may be attached and then removed (e.g., for sample removal) as desired. According to another exemplary embodiment, a user may cut a window laterally through a wall of the needle 102 to retrieve the tissue sample 102. In yet another embodiment, the system 100 further comprises a stylet sized and shaped to be received within the lumen 110 of the needle 102 so that stylet may be moved distally with respect to the needle 102 to push the tissue sample 12 distally past the flaps 104 and out of the lumen 110.

According to an exemplary surgical technique using the system 100, the needle 102 is inserted to a target tissue 10 in a living body via a working channel of an endoscope or other insertion instrument (inserted, for example, through a body lumen accessed via a natural bodily orifice). As shown in FIG. 1, the flaps 104 are biased toward the closed configuration so that the flaps remain in the closed configuration until the needle 102 is inserted into the target tissue 10. When the endoscope or insertion instrument has been advanced to a target site adjacent to target tissue 10, the needle 102 is advanced distally out of the endoscope so that the distal end 108 of the needle 102 is inserted into the target tissue 10 until a tissue sample 12 pushes the flaps 104 proximally, from the closed configuration toward the open configuration, to be received within the lumen 110, as shown in FIG. 2. In one embodiment, the needle 102 may be inserted to the target tissue 10 with a stylet received within the lumen 110 thereof so that the flaps 104 are prevented from being inadvertently moved to the open configuration until the desired target tissue 10 has been reached.

As described above, in the open configuration, the flaps 104 are folded into the recess 124 to form a sharp cutting edge at the distal end 108, which cuts tissue as the distal end 108 is inserted into the target tissue 10. Once a sufficient tissue sample 12 has been received in the lumen 110, the user draws the needle 102 proximally relative to the target tissue 10, causing the flaps 104 to return under their natural bias toward the closed configuration. As shown in FIG. 3, however, the flaps 104 are prevented from moving completely back to the closed configuration because of the volume constraints of the tissue sample received therebetween. Moving the needle 102 further proximally, however, causes the second ends 114 of the flaps 104 to cut into the tissue sample 12, severing the tissue sample 12 from surrounding target tissue 10, as shown in FIG. 4. The flaps 104 then revert to the closed configuration under their natural bias, as shown in FIG. 5, trapping the tissue sample 12 in the lumen 110, proximally of the flaps 104.

Upon removing the needle 102 from the body, the tissue sample 12 is removed from the lumen 110 using the tissue removal tool 122. In particular, the tissue removal tool 122 is mounted over the distal end 108 of the needle 102 so that the distal end 108 is received within the recess 134 at the proximal end 128 thereof. As the tissue removal tool 122 is mounted over the distal end 108, the arms 136 move the flaps 104 from the closed configuration to the open configuration, holding the flaps 104 in the open configuration so that the tissue sample 12 may be removed via the channel 132.

Although the exemplary surgical technique describes the use of the tissue removal tool 122, it will be understood by those of skill in the art that the tissue sample 12 may be removed from the lumen 110 of the needle 102 using any of a variety of methods. For example, the tissue sample 12 may be removed by detaching a distal portion of the needle 102 (including the flaps 104) from a remaining portion thereof, using a stylet to push the tissue sample 12 distally past the flaps 104, and/or cutting a window through a wall of the needle 102 to retrieve the tissue sample 12. In another embodiment, one or more of the hinges 116 may be bent or broken to move one or more of the flaps 104 from the lumen 110.

As shown in FIG. 8, a needle 202 according to another exemplary embodiment may be substantially similar to the needle 102, as described above, comprising flaps 204 extending radially inward from a distal portion of a needle body 206. Similarly to the needle body 106, the needle body 206 extends longitudinally from a proximal end (not shown) to a distal end 208 and includes a lumen 210 extending therethrough. The flaps 204, however, are not positioned at the distal end 208, but slightly proximally of the distal end 208 along a distal portion of the needle body 206. The flaps 204 are formed via a tab cut out of a wall defining the needle body 206 and bent inward toward a centerline of the needle 202. Similarly to the needle 102, the flaps 204 are biased toward a closed configuration and are bent proximally to an open configuration as the distal end 208 of the needle is inserted into target tissue.

In one exemplary embodiment, two tabs may be cut on opposing sides of the needle body 208 to form two flaps 204. It will be understood by those of skill in the art, however, that the needle 202 may be formed with any number of flaps 204. Where more than one flap 204 is included, tabs are cut about an interior circumference of the lumen 210 so that each of the flaps 204 is positioned substantially along the same point along a length of the needle body 206. The needle 202 may be utilized in substantially the same manner as described above in regard to the needle 102.

As shown in FIG. 9, a needle 302 according to another exemplary embodiment of the present disclosure is substantially similar to the needle 202 described above comprising a needle body 306 and flaps 304 coupled thereto. The needle 302, however, further comprises an oversheath 330 longitudinally slidable over the needle body 306 to move the flaps 304 between closed and open configurations. The flaps 304 are coupled to a distal portion of the needle body 306 via a hinge 316 about which the flaps 304 pivot between the closed and open configurations. In the closed configuration, each flap 304 is angled with respect to a longitudinal axis of the needle 302 such that a proximal portion 314 of the flap 304 extends into a lumen 310 of the needle body 306 to block the lumen 310 while a distal portion 312 extends outwardly past an exterior surface 332 of the needle body 306. In the open configuration, the flaps 304 extend substantially parallel to the longitudinal axis of the needle 302 such that the lumen 310 is open to receive a tissue sample therewithin.

The flaps 304 are biased toward the closed configuration so that moving the oversheath 330 distally over the needle body 306 until a distal end 334 of the oversheath abuts a portion of the flaps 304 pivots the flaps 304 about the hinge 316 from the closed configuration to the open configuration. Once the target tissue sample has been received within the lumen 310, the oversheath 330 is moved proximally with respect to the needle body 306 to permit the flaps 304 to return toward the biased closed configuration. Similarly to the needles 102, 202, moving the needle 302 proximally relative to the target tissue causes an end 313 of the proximal portion 314 extending into the lumen 310 to cut the tissue sample from a surrounding tissue to trap the tissue sample within the lumen 310, proximally of the flaps 304. It will be understood by those of skill in the art that the needle 302 may be used in substantially the same manner as described above in regard to the needles 102, 202. Rather than relying on the tissue itself to move the flaps 304 from the closed to open configuration, the flaps 304 may be toggled between the open and closed configurations via the oversheath 330. In addition, once the needle 302 has been removed from the body, the oversheath 330 may simply be moved distally relative to the needle body 306 to move the flaps 304 to the open configuration and remove the tissue sample from within the lumen 310.

It will be apparent to those skilled in the art that variations can be made in the structure and methodology of the present disclosure, without departing from the scope of the disclosure. Thus, it is intended that the present disclosure cover the modifications and variations of this disclosure provided that they come within the scope of the appended claims and their equivalents.

1-15. (canceled)

16. A device for obtaining a tissue sample, comprising:
a needle body extending along a longitudinal axis from a proximal end to a distal end and including a lumen extending therethrough; and
a plurality of flaps coupled to the needle body via hinges, each of the plurality of flaps movable between a closed configuration in which a cutting end of each of the flaps extends towards the longitudinal axis of the needle body so that the plurality of flaps extend radially inward to at least partially cover a distal opening of the lumen, and an open configuration, in which the plurality of flaps are rotated about the hinges so that cutting ends thereof are moved toward an interior surface of the lumen to uncover the distal opening of the lumen and permit a target tissue to be received therebetween and into the lumen.

17. The device of claim 16, wherein the plurality of flaps is biased toward the closed configuration.

18. The device of claim 16, wherein the hinges are living hinges.

19. The device of claim 16, wherein the plurality of flaps is coupled to the distal end of the needle body.

20. The device of claim 16, wherein the interior surface of the lumen includes a recess extending therein, the recess sized and shaped to receive one of the plurality of flaps, when the plurality of flaps is in the open configuration.

21. The device of claim 20, wherein the recess is a circumferential groove sized to receive all of the plurality of flaps, when the plurality of flaps is in the open configuration.

22. The device of claim 16, wherein the cutting end of each of the plurality of flaps is sharpened to cut the target tissue from a surrounding tissue, trapping the target tissue in the lumen.

23. The device of claim 16, wherein the plurality of flaps is formed via a tab cut through a wall defining the needle body and bent toward the longitudinal axis of the needle body.

24. The device of claim 16, wherein the plurality of flaps is pivotally coupled to a distal portion of the needle body.

25. The device of claim 16, further comprising:
an oversheath longitudinally slidable over the needle body to move the plurality of flaps between the closed and open configurations.

26. A system for collecting a tissue sample, comprising:
a needle extending longitudinally from a proximal end to a distal end, the needle including a lumen extending therethrough and a plurality of flaps coupled thereto and movable between a closed configuration and an open configuration, each of the plurality of flaps extending from a first end coupled to the distal end via a hinge biasing the plurality of flaps in the closed configuration to a second end, the second end extending toward a longitudinal axis of the needle so that the plurality of flaps extend radially inward to cover a distal opening of the lumen in the closed configuration, the plurality of flaps adapted for rotating about the hinges via which the plurality of flaps is connected to the needle body so that second ends thereof are moved toward an interior surface of the lumen to uncover the distal opening of the lumen and permit a target tissue to be received therebetween in the open configuration; and
a tissue removal tool coupleable to the distal end of the needle to remove the target tissue from within the lumen of the needle, the tissue removal tool including arms at a proximal end thereof for holding the plurality of flaps in the open configuration when the tissue removal tool is coupled to the needle.

27. The system of claim 26, wherein the tissue removal tool includes a channel extending therethrough, the channel substantially aligning with the lumen of the needle when the tissue removal tool is coupled thereto.

28. The system of claim 26, wherein the plurality of flaps is coupled to the distal end of the needle via a living hinge.

29. The system of claim 26, wherein the interior surface of the lumen includes a recess extending therein, the recess sized and shaped to receive at least one of the plurality of flaps, when the plurality of flaps is in the open configuration.

30. The system of claim 26, wherein the cutting ends of the plurality of flaps is sharpened to cut the target tissue from a surrounding tissue, trapping the target tissue in the lumen.

31. A method for collecting a tissue sample, comprising:
inserting a needle to a target tissue via a working channel of an endoscope, the needle extending longitudinally from a proximal end to a distal end and including a lumen extending therethrough;
inserting the distal end of the needle into the target tissue such that a portion of the target tissue moves a plurality of flaps coupled to a distal portion of the needle from a closed configuration, in which cutting ends of the plurality of flaps extend toward a longitudinal axis of the needle to cover a distal opening of the lumen, to an open configuration, in which the plurality of flaps rotate about hinges connecting the plurality of flaps to the needle to uncover the distal opening and permit the target tissue to be received therein; and
moving the needle proximally relative to the target tissue such the plurality of flaps is moved toward the closed configuration and the cutting ends of the plurality of flaps cut the portion of the target tissue received within the lumen from a surrounding portion of the target tissue, trapping a tissue sample therein.

32. The method of claim 31, further comprising:
removing the tissue sample from the lumen of the needle by coupling a tissue removal tool to the distal end of the needle, the tissue removal tool including arms holding the plurality of flaps in the open configuration to remove the tissue sample via a channel thereof.

33. The method of claim 31, further comprising:
removing the tissue sample from the lumen of the needle by detaching the distal portion of the needle from a remaining portion thereof.

34. The method of claim 31, further comprising:
removing the tissue sample from the lumen of the needle by cutting a window laterally through a wall of the needle.

35. The method of claim 31, wherein the plurality of flaps is coupled to the distal end of the needle via a living hinge and an interior surface of the lumen immediately proximal of the plurality of flaps includes a recess for receiving at least one of the plurality of flaps in the open configuration so that a distal edge of the needle is sharpened to cut the target tissue as the needle is inserted therein.
